(19) United States
(12) Patent Application Publication
(10) Pub. No.: US 2025/0309911 A1
(43) Pub. Date: Oct. 2, 2025

(54) BIOSIGNAL DETECTION DEVICE, HYBRID ANALOG DIGITAL CONVERTERS AND ANALOG DIGITAL CONVERTERSION METHOD FOR BIOSIGNAL

(71) Applicant: Electronics and Telecommunications Research Institute, Daejeon (KR)

(72) Inventors: Kisu KIM, Daejeon (KR); Bon Tae Koo, Daejeon (KR); Yong Hee Kim, Daejeon (KR); Sang Don Jung, Daejeon (KR)

(73) Assignee: Electronics and Telecommunications Research Institute, Daejeon (KR)

(21) Appl. No.: 19/090,861

(22) Filed: Mar. 26, 2025

(30) Foreign Application Priority Data
Mar. 26, 2024 (KR) 10-2024-0041127
Mar. 13, 2025 (KR) 10-2025-0032646

(51) Int. Cl. H03M 1/38; A61B 5/00; H03K 17/00

(52) U.S. Cl. CPC H03M 1/38; A61B 5/7225; H03K 17/002

(57) ABSTRACT

A bio-signal detection apparatus includes an analog front end unit includes a plurality of analog front end circuits that process a detected signal; an analog-to-digital conversion unit that converts an output signal of the analog front end unit to a digital code and a multiplexer (MUX) that outputs a processed signal by the analog front end circuits to the analog-to-digital conversion unit; wherein the analog digital conversion unit comprises: a comparator, a successive-approximate analog-to-digital converter (SAR ADC) that converts output signal of the analog front end circuit and form MSB side j-bit of the digital code, and a single slope ADC that converts the output signal of the analog front end circuit to k bit of the digital code.

### CROSS-REFERENCE TO RELATED APPLICATION

This application claims priority to and the benefit of Korean Patent Application Nos. 10-2024-0041127, filed on Mar. 26, 2024 and 10-2025-0032646, filed on Mar. 13, 2025, the disclosures of which are incorporated herein by reference in its entirety.

### BACKGROUND

### 1. Field of the Invention

The present disclosure generally relates to bio-signal detection devices, hybrid analog-to-digital converters, and analog-to-digital conversion methods for bio-signals.

### 2. Discussion of Related Art

The BCI (Brain Computer Interface) system has been developed based on the demonstration of brain-controlled BCI in 2004, in which a patient with a spinal cord injury moved the cursor of a computer by thinking, by implanting a neural electrode into the patient's brain. Based on this development, closed-loop interfaces between brain-computer systems are being studied.

With the recent development of semiconductor processes, it has become possible to develop such brain-computer interface systems that are designed as semiconductor integrated circuits and are completely inserted inside the body. In order to manufacture a fully-inserted type devices, it is necessary to detect low signal levels in an environment with many noise elements at the μV to mV level, and it is essential to miniaturize the core block through optimization, achieve ultra-low power consumption, and manage power, and to collect neuronal signals more precisely through channel expansion.

### SUMMARY

According to an aspect of the present embodiment, there is provided a bio-signal detection apparatus comprising: an analog front end unit comprising a plurality of analog front end circuits that process a detected signal, an analog-to-digital conversion unit that converts an output signal of the analog front end unit to a digital code and a multiplexer (MUX) that outputs a processed signal by the analog front end circuits to the analog-to-digital conversion unit, wherein the analog digital conversion unit comprises: a comparator, a successive-approximate analog-to-digital converter (SAR ADC) that converts output signal of the analog front end circuit and form MSB side j-bit of the digital code, and a single slope ADC that converts the output signal of the analog front end circuit to k bit of the digital code.

In one aspect of the present embodiment, the analog front end unit comprises: a low-noise amplifier (LNA) and a bandpass filter (BPF) that passes a band including a target bio-signal. According to the present aspect, the bio-signal detection apparatus further comprises: a sample and hold unit that samples and holds the output signal of the band pass filter.

In one aspect of the present embodiment, the SAR ADC comprises: a capacitor array including a plurality of capacitors, the capacitor's capacitances are in power of two, and one electrode of each capacitor is connected to the comparator, other electrode of each capacitor is connected to a switch and selectively provided with any one of an output signal, a reference voltage and a ground voltage and the capacitor array further comprises a ramp capacitor, that has smallest capacitance of capacitors in the capacitor array. According to the present aspect, the single slope ADC comprises a ramp signal generator that forms a ramp signal which increases in a single slope, and the ramp signal is provided to the other electrode of the ramp capacitor via the switch.

In one aspect of the present embodiment, the SAR ADC and the single slope ADC shares the comparator.

In one aspect of the present embodiment, the single slope ADC outputs the k bit of digital code that corresponds to a time that a magnitude of linearly increasing voltage exceeds magnitude of the output signal. According to the present aspect, the single slope ADC comprises the comparator, and a counter that counts clock and wherein the counter starts counting when the comparator output is in a first state, and the counter stops counting when the comparator output transits to a second state, and the counter outputs the counted value as the k bit of the digital code.

In one aspect of the present embodiment, the plurality of the bio-signal detection apparatus shares one ramp signal generator.

According to an aspect of another embodiment, there is provided a hybrid analog-to-digital converter comprising: a comparator; a successive-approximate analog-to-digital converter (SAR ADC) that converts an analog signal to a j bit digital code; a single slope ADC that converts the analog signal to a k bit digital code and a register that saves the j bit digital code and the k bit digital code; wherein the SAR ADC and the single slope ADC share the comparator.

In one aspect of the present embodiment, the hybrid analog-to-digital converter further comprises: a sample and hold unit that samples and holds the analog signal.

In one aspect of the present embodiment, the SAR ADC comprises: a capacitor array including a plurality of capacitors, the capacitor's capacitances are in power of two, and one electrodes of each capacitor is connected to the comparator, other electrode of each capacitor is connected to a switch and selectively provided with any one of an output signal, a reference voltage and a ground voltage and the capacitor array further comprises a ramp capacitor, that has smallest capacitance of capacitors in the capacitor array.

According to the present aspect, the single slope ADC comprises a ramp signal generator that forms a ramp signal which increases with single slope, and the ramp signal is provided to the other electrode of the ramp capacitor via the switch.

In one aspect of the present embodiment, the single slope ADC outputs the k bit of digital code that corresponds to a time that a magnitude of linearly increasing voltage exceeds magnitude of the output signal.

According to the present aspect, the single slope ADC comprises: the comparator, and

a counter that counts clock and wherein the counter starts counting when the comparator output is in the first state, and the counter stops counting when the comparator output transits to the second state, and the counter outputs the counted value as the k bit of the digital code.

According to an aspect of still another embodiment, there is provided a bio-signal analog-to-digital conversion method comprising steps of: analog signal processing the bio-signal; converting the processed bio-signal to MSB side j bit digital code by a successive-approximate analog-to-digital converter (SAR ADC); converting the bio-signal to a k-bit digital code adjacent to the j bit digital code by a single slope ADC which includes a ramp signal generator; wherein the converting the bio-signal to a k-bit digital code is performed by forming the k-bit digital code that corresponds to a time of a linearly increasing voltage exceeds the output signal.

In one aspect of the present embodiment, the SAR ADC comprises: a capacitor array including a plurality of capacitors, the capacitor's capacitances are in a power of two, and one electrode of the capacitors is connected to the comparator, other electrode of the capacitor is connected to a switch and selectively provided with any one of an output signal, a reference voltage and a ground voltage and the capacitor array further comprises a ramp capacitor, that has smallest capacitance of capacitors in the capacitor array.

In one aspect of the present embodiment, the converting the bio-signal to k-bit digital code further includes: forming a linearly increasing voltage from reference voltage, by providing the ramp signal formed by the ramp signal generator to the ramp capacitor, wherein the reference voltage corresponds to the j bit of the digital code; forming the k bit of the digital code by counting the clocks until the magnitude of the signal increasing from the reference voltage and magnitude of the bio-signal reverses.

### BRIEF DESCRIPTION OF DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating an overview of the bio-signal detection device according to the present embodiment.

FIG. 2 is a block diagram illustrating an overview of the analog front-end circuit included in the analog front-end unit.

FIG. 3 is a block diagram illustrating an overview of the hybrid analog-to-digital converter unit of this embodiment.

FIG. 4 is an overview diagram to explain the operation of the successive-approximation ADC.

FIGS. 5A to 5D are overview diagrams to explain the operation of the successive-approximation ADC according to an embodiment.

FIG. 6 is a schematic diagram of the state of a single-slope ADC after the successive-approximation ADC finished a digital conversion of the MSB-side j-bit of the signal.

FIGS. 7A to 7E are schematic timing diagrams of signals to explain the operation of a single-slope ADC.

FIG. 8 is a schematic diagram showing an overview of a bio-signal detection module including the bio-signal detection devices.

### DETAILED DESCRIPTION OF EXEMPLARY EMBODIMENTS

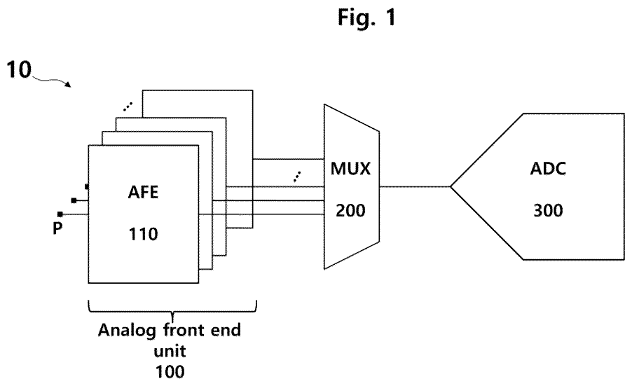

Hereinafter, the present embodiment will be described with reference to the accompanying drawings. FIG. 1 is a schematic diagram of bio-signal detection apparatus according to present embodiment. The bio-signal detection apparatus (10) comprises: an analog front end unit 100 comprising a plurality of analog front end circuits 110 that process a detected signal; an analog-to-digital conversion unit 300 that converts an output signal of the analog front end unit to a digital code and a multiplexer (MUX) 200 that outputs a processed signal by the analog front end circuits to the analog-to-digital conversion unit; wherein the analog digital conversion unit 300 comprises: a comparator, successive-approximate analog-to-digital converter (SAR ADC) that converts output signal of the analog front end circuit and form MSB side j-bit of the digital code, and a single slope ADC that converts the output signal of the analog front end circuit to k bit of the digital code.

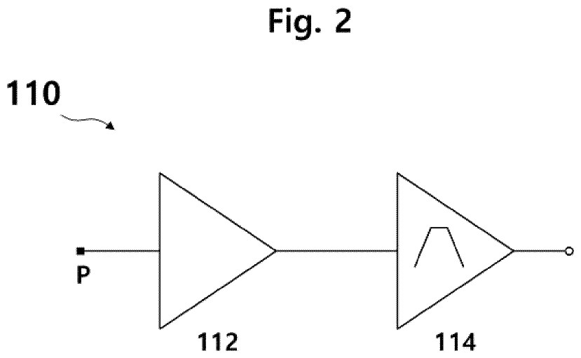

FIG. 2 is a block diagram that exemplifies an overview of the analog front-end circuit 110 included in the analog front-end unit 100. Referring to FIGS. 1 and 2, the analog front-end unit 100 includes a plurality of analog front-end circuits 110, and each of the analog front-end circuits 110 includes a probe P for collecting a bio-signal. In one embodiment, probes P can be inserted and implanted in the body, such as the brain, spinal cord, and nervous system, to collect biological signals, such as neural signals.

The bio-signal collected by the probe P is amplified by a low-noise amplifier (LNA) 112. The low-noise amplifier 112 suppresses the intrusion of noise and amplifies the bio-signal collected by the probe P to a desired gain and outputs the amplified signal.

A band pass filter (BPF) 114 passes the signal of the desired band from the signal output by the low noise amplifier 112 and outputs it. The pass band of the band pass filter 114 may vary depending on the type of signal to be obtained.

The analog front-end unit 100 includes a plurality of analog front circuits 110, and signals output by the plurality of analog front circuits 110 are input to the hybrid analog-to-digital converter 300 of the present embodiment through a multiplexer (MUX).

According to an embodiment not illustrated, the analog front-end unit may further include a sample-and-hold unit that samples the signal provided by the multiplexer and holds the sample. According to another embodiment not illustrated, the analog-to-digital converter may further include sample and hold unit that samples the signal provided by the multiplexer and holds the sample.

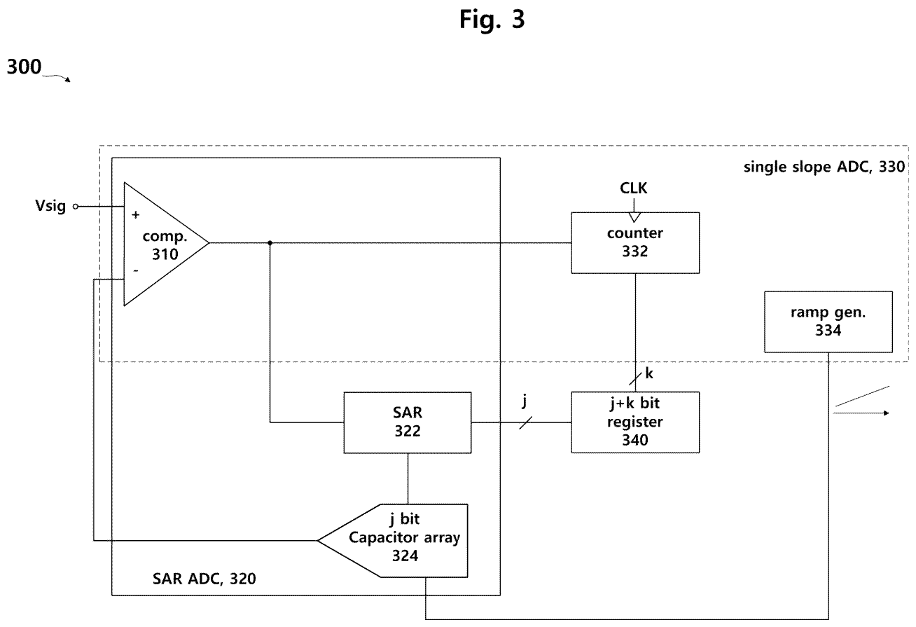

FIG. 3 is a block diagram illustrating an overview of the hybrid analog-to-digital converter 300 of the present embodiment and FIG. 4 is an overview diagram to explain the operation of the successive-approximation ADC. Referring to FIGS. 3 and 4, the hybrid analog-to-digital converter 300 comprises: successive approximate (SAR) ADC 320 that converts the output signal of the analog front end into digital form to form the digital code of the j-bit on the MSB side, and a single-slope ADC 330 that converts the output signal of the analog front end into digital form to form the digital code of the subsequent k-bit.

The comparator 310 compares the processed output signal Vsig output by the analog front-end unit 110 with the first reference voltage Vref1 output by the capacitor array 324. In one embodiment, the first reference voltage Vref0 may be a 50% voltage of the reference voltage Vref. The comparator 310 outputs logic 1 when the output signal Vsig is greater than the first reference voltage Vref0 and outputs logic 0 when the output signal Vsig is less than the first reference voltage Vref0. In the example shown, the comparator 310 outputs logic 1 because the output signal Vsig is greater than the first reference voltage Vref0.

According to the control signal of the SAR controller 322, the capacitor array 324 to which the output of the comparator 310 is provided outputs a second reference voltage Vref1 to perform binary search following the first reference voltage Vref0. The capacitor array 324 outputs the second reference voltage Vref1 at 75% Vref, which is half the difference between the maximum voltage Vref and the first reference voltage Vref0, and the comparator 310 outputs the result of comparing the output signal Vsig with the second reference voltage Vref1. In the illustrated example, the output signal Vsig is less than the second reference voltage Vref1, so the comparator 310 outputs a zero.

The capacitor array 324 to which the output of the comparator 310 is provided outputs 62.5% Vref, which is half of the difference between the first reference voltage Vref0 and the second reference voltage, as the third reference voltage Vref2. The comparator 310 compares the output signal Vsig with the third reference voltage Vref2 and outputs the result of the comparison. In the example shown, the output signal Vsig is greater than the third reference voltage Vref2, so the comparator 310 outputs 1. The voltage output by the analog front end can be digitally converted using the example of the successive-approximate digital-to-analog converter. In one embodiment, the successive-approximation ADC 320 forms a comparison result for the input signal Vsig and outputs it to the register 340. The comparison result may be a j-bit, for example, when the input signal Vsig is to be converted into a total of 10-bit digital code, the successive-approximation ADC 320 can output a digital code of 2 to 6 bits on the MSB side.

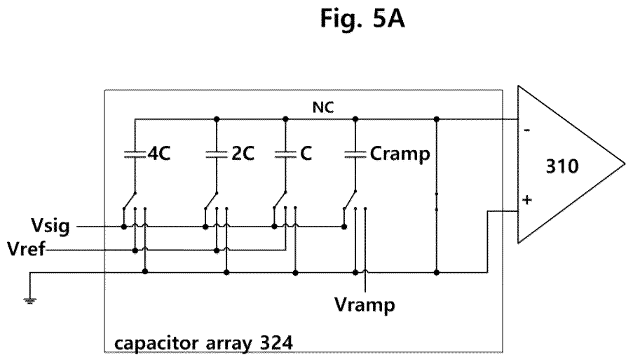

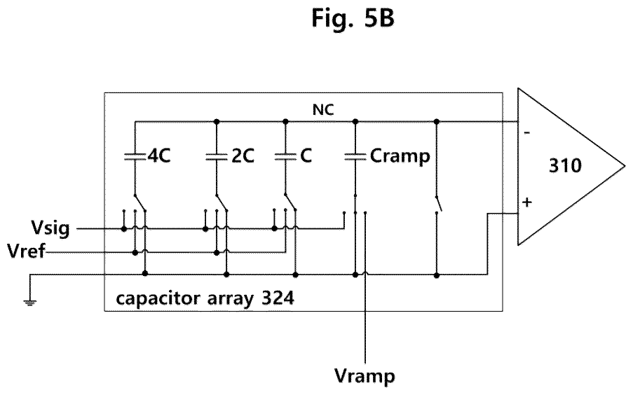

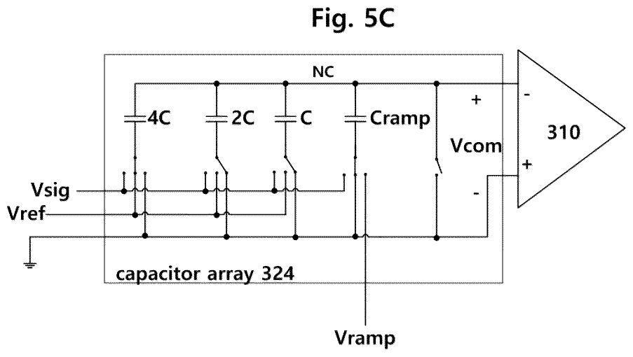

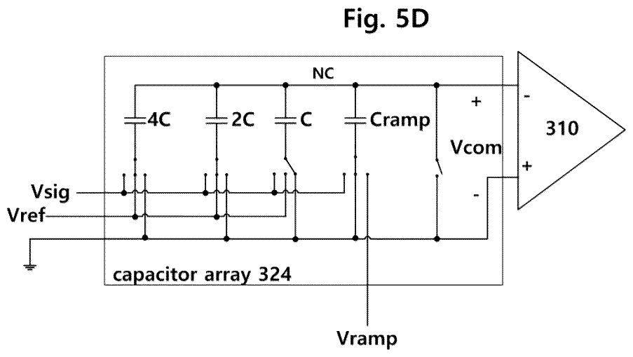

FIGS. 5A to 5D are schematic drawings to illustrate the operation of the SAR ADC 320 according to an embodiment. SAR ADC 310 of the embodiment illustrated in FIG. 5A is a SAR ADC using charge sharing. Referring to FIG. 5A, the SAR ADC 310 of present embodiment includes a capacitor array comprising at least j capacitors having a capacitance of a power of two and a ramp capacitor Cramp. The capacitance of the ramp capacitor Cramp is equal to the smallest capacitance in the capacitor array.

As illustrated in FIG. 5A, the common node NC of the capacitor array is connected to the reference potential by the switch, and the output signal Vsig is supplied to the capacitors 4C, 2C, C, Cramp by the switch, so that the capacitors 4C, 2C, C, Cramp are charged with the charges corresponding to the voltage of the output signal Vsig.

The capacitors included in the capacitor array 324 are connected to the switch. The switch may be a MOSFET transistor switch whose conduction and blocking are controlled by the control signal provided by the SAR control unit 322.

Referring to FIG. 5B, the switches are controlled by the control signal provided by the SAR control unit 322, and each capacitor is connected to the ground potential. In addition, the switch connected between the inverting input and non-inverting input of the comparator 310 is blocked, and capacitors remain charged with the charge corresponding to the output signal Vsig.

Then, as illustrated in FIG. 5C, the lower electrode of the MSB capacitor 4C is connected to the reference potential Vref, causing redistribution of charge between the capacitors, and the potential at the common node NC is formed as Vcom=−Vsig+0.5 Vref. Therefore, a comparison is performed between the input voltage Vsig and 50% of Vref. The comparator 310 outputs a logic 1 if Vsig>0.5 Vref and a logic 0 if Vsig<0.5 Vref. As illustrated in FIG. 4, since Vsig is greater than Vref0, which is 0.5 Vref, the comparator 310 outputs a logic 1.

The SAR control unit 322 receives an output signal from the comparator 310 indicating that Vsig is greater than Vref0, which is 0.5 Vref, and provides a switch control signal to the capacitor array 324 to compare Vsig to 0.75 Vref. The SAR control unit 322 provides a control signal so that the MSB capacitor 4C and the lower capacitor 2C adjacent to the MSB capacitor 4C are connected to Vref as illustrated in FIG. 5D.

As the lower electrode of the capacitor 2C is connected to Vref, the charge in the capacitor is redistributed, and the potential at the common node NC is formed as Vcom=−Vsig+0.75Vref. Therefore, the comparator 310 compares Vsig with +0.75 Vref and outputs a logic 0 because Vsig is less than Vref1, which is 0.75 Vref.

However, according to an unillustrated embodiment, when Vsig<0.5 Vref and the output of the comparator is logic 0, the bottom electrode of the MSB capacitor is connected to ground again and the bottom electrode of the adjacent lower capacitor 2C is connected to Vref, and the charge in the capacitor is redistributed. Accordingly, the potential of the common node is formed as Vcom=−Vsig+0.25Vref. Therefore, the comparator compares Vsig with +0.25Vref and outputs a value corresponding to the result of comparing Vsig with 0.25Vref.

The results of the digital conversion of the MSB-side j-bit of Vsig by the SAR ADC 320 in comparison with the Vsig are binary 101, as shown in FIG. 4, and they are stored in the register 340 (see FIG. 3).

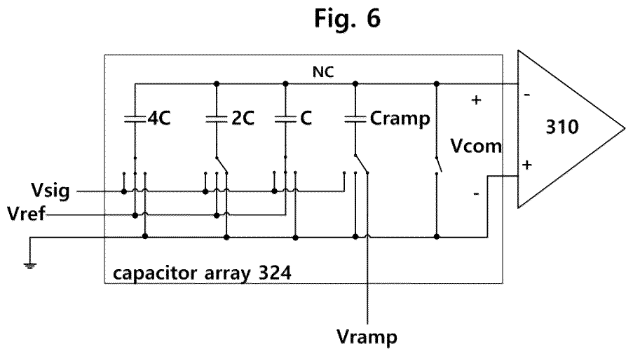

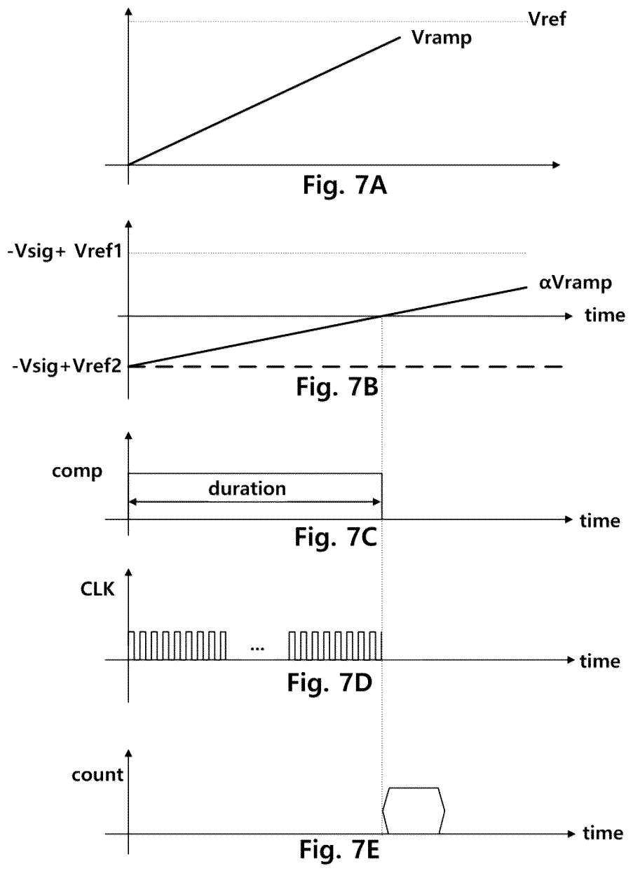

FIG. 6 is a schematic illustration of the state when the single-slope ADC 330 operates after the SAR ADC 320 performs the digital conversion of the MSB side j-bit of the signal Vsig. FIGS. 7A to 7D are schematic timing diagrams of the signals to explain the operation of the single-slope ADC 330. Referring to FIGS. 3, 6 and 7, when the SAR ADC 320 completes the MSB j-bit digital conversion for the Vsig signal, the single-slope ADC 330 subsequently performs the k-bit digital conversion.

When the SAR ADC 320 has completed digital conversion, the ramp capacitor Cramp is charged with the charge corresponding to Vref2, which is compared to Vsig during the operation of the SAR ADC 320.

FIG. 7A is a drawing that illustrates an overview of the ramp signal Vramp that the ramp signal generator 334 forms and provides. As illustrated in FIG. 7A, the ramp signal generator 334 generates and outputs a ramp signal Vramp that increases linearly from 0 to Vref from the point at which the single-slope ADC 330 is operating.

FIG. 7B is a diagram illustrating the voltage Vcom that is formed between the inverting input and the non-inverting input of the comparator 310. The voltage formed between the inverting input and non-inverting input can be expressed as Vcom=−Vsig+Vref2+αVramp. The voltage that makes up Vcom corresponds to the sum of −Vsig+Vref2 and αVramp, as shown in FIG. 7B.

The slope of the αVramp signal applied to the ramp capacitor Cramp is lowered by α (0<α<1) times compared to the slope of the ramp signal Vramp as the Vramp signal is voltage divided by the capacitors 4C, 2C, C, Cramp. In the illustrated embodiment, αcan be ⅛. For example, the coefficient of the Vramp signal may vary depending on the number of capacitors included in the capacitor array. The Vcom voltage formed between the inverting and non-inverting inputs of the comparator 310 increases linearly with the αVramp slope from the voltage sum of −Vsig and Vref2.

FIG. 7C is a schematic illustration of the comparison signal comp output by the comparator 310. Referring to FIG. 7C, the comparator 310 compares the magnitude of the Vcom and the ground voltage and outputs the comparison result value.

If Vcom<0, −Vsig+Vref2+αVramp<0. Therefore, αVramp<Vsig−Vref2, which indicates that the magnitude of αVramp is smaller than the magnitude of the Vsig voltage from Vref2, thus, the output of the comparator 310 is logic 1.

On the other hand, if Vcom>0, −Vsig+Vref2+αVramp>0. Therefore, αVramp>Vsig−Vref2, which indicates that the magnitude of αVramp is larger than the magnitude of the Vsig voltage from Vref2, and thus, the output of the comparator 310 is logic 0, and the comparison signal comp output by the comparator 310 forms a falling edge. In addition, the duration of the comparison signal comp, which is the time from when the comparator 310 transitions its output from logic 1 to logic 0 to output the falling edge, is proportional to the magnitude of the Vsig signal.

As shown in FIGS. 7D and 7E, counter 332 measures the duration of the comparison signal comp by counting the input clock signal CLK from the time when the single-slope ADC 330 starts to operate until the time when the comparison signal comp transitions from the logic 1 state to the logic 0 state, and outputs the count result to register 340. Therefore, the register 340 stores MSB side j bits output from the SAR ADC 320 and the subsequent k bits converted by the single-slope ADC 330.

By providing a ramp signal to the end of the ramp capacitor Cramp, it is possible to convert the charge remaining in the ramp capacitor Cramp, which could not be converted by the SAR ADC, into a digital signal.

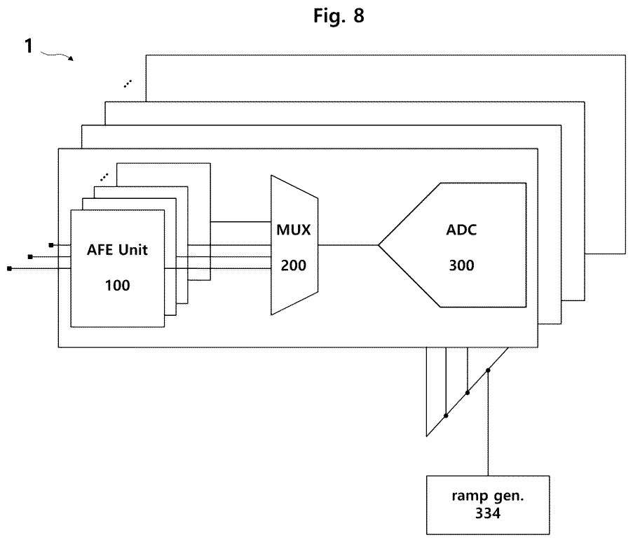

FIG. 8 is a schematic drawing showing an overview of the bio-signal detection module 1 including the bio-signal detection devices 10 illustrated. As shown in FIG. 8, multiple bio-signal detection devices 10 can form a bio-signal detection module 1. The bio-signal detection module 1 has the advantage of being able to share a single lamp signal generator 334, thereby reducing power consumption and footprint.

According to the embodiment described above, the advantages of the SAR ADC, which consumes low power and occupies a relatively small area, and the advantages of the single-slope ADC, which has relatively high precision and strong noise characteristics, can both be obtained.

Furthermore, the advantage of being able to reduce the area occupied and the conversion time by converting some of the j+k bits to SAR ADCs and single-slope ADCs is also provided.

Although the present disclosure has been described with reference to embodiments illustrated in the accompanying drawings in order to help the understanding of the present disclosure, this is only an exemplary embodiment for implementation, and those of ordinary skill in the art will understand that various modifications and other equivalent embodiments are possible therefrom. Accordingly, the true technical scope of the present disclosure is to be determined from the spirit of the appended claims.

What is claimed is:

1. A bio-signal detection apparatus comprising:
an analog front end unit comprising a plurality of analog front end circuits that process a detected signal;
an analog-to-digital conversion unit that converts an output signal of the analog front end unit to a digital code and
a multiplexer (MUX) that outputs a processed signal by the analog front end circuits to the analog-to-digital conversion unit;
wherein the analog digital conversion unit comprises:
a comparator,
a successive-approximate analog-to-digital converter (SAR ADC) that converts output signal of the analog front end circuit and form MSB side j-bit of the digital code, and
a single slope ADC that converts the output signal of the analog front end circuit to k bit of the digital code.

2. The bio-signal detection apparatus of claim 1, wherein the analog front end unit comprises:
a low-noise amplifier (LNA) and
a bandpass filter (BPF) that passes a band including a target bio-signal.

3. The bio-signal detection apparatus of claim 2, wherein the bio-signal detection apparatus further comprises:
a sample and hold unit that samples and holds the output signal of the band pass filter.

4. The bio-signal detection apparatus of claim 1, wherein the SAR ADC comprises:
a capacitor array including a plurality of capacitors, the capacitor's capacitances are in power of two, and one electrode of each capacitor is connected to the comparator,
other electrode of each capacitor is connected to a switch and selectively provided with any one of an output signal, a reference voltage and a ground voltage and
the capacitor array further comprises a ramp capacitor, that has smallest capacitance of capacitors in the capacitor array.

5. The bio-signal detection apparatus of claim 4, wherein the single slope ADC comprises a ramp signal generator that forms a ramp signal which increases in a single slope, and the ramp signal is provided to the other electrode of the ramp capacitor via the switch.

6. The bio-signal detection apparatus of claim 1, wherein the SAR ADC and the single slope ADC shares the comparator.

7. The bio-signal detection apparatus of claim 1, wherein the single slope ADC outputs the k bit of digital code that corresponds to a time that a magnitude of linearly increasing voltage exceeds magnitude of the output signal.

8. The bio-signal detection apparatus of claim 7, wherein the single slope ADC comprises the comparator, and
a counter that counts clock and
wherein the counter starts counting when the comparator output is in a first state, and the counter stops counting when the comparator output transits to a second state, and the counter outputs the counted value as the k bit of the digital code.

9. The bio-signal detection apparatus of claim 4, wherein the plurality of the bio-signal detection apparatus shares one ramp signal generator.

10. A hybrid analog-to-digital converter comprising:
a comparator;
a successive-approximate analog-to-digital converter (SAR ADC) that converts an analog signal to a j bit digital code;
a single slope ADC that converts the analog signal to a k bit digital code and
a register that saves the j bit digital code and the k bit digital code;
wherein the SAR ADC and the single slope ADC share the comparator.

11. The hybrid analog-to-digital converter of claim 10, wherein the hybrid analog-to-digital converter further comprises:
a sample and hold unit that samples and holds the analog signal.

12. The hybrid analog-to-digital converter of claim 10, wherein the SAR ADC comprises:
a capacitor array including a plurality of capacitors, the capacitor's capacitances are in power of two, and one electrodes of each capacitor is connected to the comparator,
other electrode of each capacitor is connected to a switch and selectively provided with any one of an output signal, a reference voltage and a ground voltage and
the capacitor array further comprises a ramp capacitor, that has smallest capacitance of capacitors in the capacitor array.

13. The hybrid analog-to-digital converter of claim 12, wherein the single slope ADC comprises a ramp signal generator that forms a ramp signal which increases with single slope, and
the ramp signal is provided to the other electrode of the ramp capacitor via the switch.

14. The hybrid analog-to-digital converter of claim 10, wherein the single slope ADC outputs the k bit of digital code that corresponds to a time that a magnitude of linearly increasing voltage exceeds magnitude of the output signal.

15. The hybrid analog-to-digital converter of claim 14, wherein the single slope ADC comprises:
the comparator, and
a counter that counts clock and
wherein the counter starts counting when the comparator output is in the first state, and the counter stops counting when the comparator output transits to the second state,
and the counter outputs the counted value as the k bit of the digital code.

16. A bio-signal analog-to-digital conversion method comprising steps of:
analog signal processing the bio-signal;
converting the processed bio-signal to MSB side j bit digital code by a successive-approximate analog-to-digital converter (SAR ADC);
converting the bio-signal to a k-bit digital code adjacent to the j bit digital code by a single slope ADC which includes a ramp signal generator;
wherein the converting the bio-signal to a k-bit digital code is performed by forming the k-bit digital code that corresponds to a time of a linearly increasing voltage exceeds the output signal.

17. A bio-signal analog-to-digital conversion method according to claim 16, wherein the SAR ADC comprises:
a capacitor array including a plurality of capacitors, the capacitor's capacitances are in a power of two, and one electrode of the capacitors is connected to the comparator,
other electrode of the capacitor is connected to a switch and selectively provided with any one of an output signal, a reference voltage and a ground voltage and
the capacitor array further comprises a ramp capacitor, that has smallest capacitance of capacitors in the capacitor array.

18. A bio-signal analog-to-digital conversion method according to claim 17, wherein the converting the bio-signal to k-bit digital code further includes:
forming a linearly increasing voltage from reference voltage, by providing the ramp signal formed by the ramp signal generator to the ramp capacitor, wherein the reference voltage corresponds to the j bit of the digital code;
forming the k bit of the digital code by counting the clocks until the magnitude of the signal increasing from the reference voltage and magnitude of the bio-signal reverses.
